(19) United States
(12) Patent Application Publication
(10) Pub. No.: US 2004/0152110 A1
(43) Pub. Date: Aug. 5, 2004

(54) Use of semaphorin 3A for diagnosing and treating cancer, especially prostate cancer

(75) Inventors: Alain Latil, Jouy En Josas (FR); Olivier Cussenot, Paris (FR); Michele Algarte-Genin, Paris (FR)

Correspondence Address: HESLIN ROTHENBERG FARLEY & MESITI PC, 5 COLUMBIA CIRCLE, ALBANY, NY, 12203, US

(73) Assignee: Urogene, Evry (FR)

(21) Appl. No.: 10/687,241

(22) Filed: Oct. 16, 2003

Related U.S. Application Data
(63) Continuation of application No. PCT/FR02/00118, filed on Jan. 11, 2002.
(60) Provisional application No. 60/284,508, filed on Apr. 19, 2001.

(51) Int. Cl. C12Q 1/68; G01N 33/574

(52) U.S. Cl. 435/6; 435/7.23

(57) ABSTRACT

The invention relates to a method for in vitro evaluation of the aggressiveness of tumor cells by measuring the level of expression of all or part of a gene coding for semaphorin 3A in healthy epithelial cells and in epithelial tumor cells. The invention also relates to a method of inhibiting the invasive power of epithelial tumor cells, consisting in increasing the levels of semaphorin 3A levels present in and/or in the vicinity of said epithelial tumor cells.

The present invention relates, in general, to the diagnosis, the evaluation and treatment of cancers, and more particularly of prostate cancer. Specifically, the present invention relates to a method for evaluating the invasive power of tumor cells and to a method for treating said tumor cells. The present invention also describes kits and compositions for carrying out said methods.

Currently, prostate cancer is, after bronchopulmonary cancer, the most widely occurring cancer in men, and more particularly in men over the age of 50 (1, 2, 3) [the numbers in bold, in brackets, refer to the attached list of bibliographical references].

Due to the size of and the increase in the number of men suffering from this cancer, the study of its development and the search for effective and relatively inexpensive treatments have, in recent times, become priorities for the medical world.

In general, three major processes contribute to the development of malignant epithelial tumors, namely proliferation, invasion and angiogenesis.

In a first step, an increase in the tumor mass occurs due to proliferation of the cancer cells and to the decrease in the physiological apoptotic processes.

Concomitantly, angiogenesis is the process which leads to the formation of blood vessels which vascularize the tumor and allow it to develop. The angiogenesis consists of the proliferation and the migration of healthy endothelial cells of the blood and lymphatic vessels located in the vicinity of the tumor, under the effect of growth factors secreted by the cancer cells.

These growth factors are, for example, the VEGFs; they act via receptors expressed at the surface of the endothelial cells, such as VEGFR-1 (flt-1) or VEGFR-2 (flk-1).

In a second step, invasion of the surrounding normal tissues leads to extension of the tumor through two concomitant mechanisms: (i) enzymatic degradation of the basal lamina and (ii) migration of the cells to the surrounding tissues. Finally, the invasion may lead to the formation of distant metastases, when the tumor cells reach the blood circulation.

The term “proliferation” should be understood to mean the rapid multiplication of the cells, said multiplication taking place by cell division at the time of mitosis.

The term “migration” should be understood to mean the ability which malignant tumor cells have to move around.

One of the approaches for finding genes involved in these various processes has consisted in determining and comparing the levels of expression of many genes in normal cells and also tumor cells so as to demonstrate differences in expression in said normal cells and tumor cells.

The genes thus identified can be used to develop diagnostic or therapeutic methods which are more targeted as a function of the stage of development of the pathological condition.

Through this approach, the inventors have identified, firstly, semaphorin-3A as a potential tumor suppressor gene and, secondly, the receptor for semaphorin-3A, neurophilin-1, as a therapeutic target for treating advanced stage epithelial tumors.

Neurophilin is a membrane receptor initially described in the cells of the nervous system and then identified in endothelial and epithelial cells. Two forms of neurophilin have been described, neurophilin-1 and neurophilin-2, having different properties. The ligands for these two forms are semaphorin-3E and -3F and also VEGF₁₆₅, while only neurophilin-1 is capable of binding semaphorin-3A with high affinity (4). The semaphorin family comprises about twenty members divided up into 8 classes. Some are soluble secreted proteins whereas others are transmembrane proteins. Class 3 is the most well characterized and comprises six members (Sema3A-F).

In neurons, neurophilin-1 is associated with membrane proteins of the plexin family (5, 6). The ligand for this heterodimeric receptor is semaphorin-3A, which brings about growth cone repulsion in development. Many experiments have shown axonal development in the opposite direction to gradients of semaphorin-3A, thus contributing to the architectural development of the nervous system (7, 8).

In blood vessel endothelial cells, neurophilin-1 is associated with receptors of the VEGFR family. The ligand for the heterodimeric receptor formed by neurophilin-1 and VEGFR2 (flk-1) is VEGF₁₆₅, and its binding to said ligand causes an increase in cell proliferation and migration, two mechanisms which contribute to the angiogenesis process, in particular during tumor development (9, 10, 11).

Finally, neurophilin-1 has also been identified in tumor cells of epithelial origin. Some studies have shown that VEGF₁₆₅ stimulates the migration of epithelial tumor cells expressing neurophilin-1. Without wishing to be bound by any theory, it has been proposed that, in these cells, neurophilin-1 acts as a single receptor for VEGF₁₆₅ (12).

On the basis of these data, several methods have been proposed, aimed at reducing tumor development by inhibition of the action of VEGF₁₆₅ on neurophilin-1.

Two methods have, for example, been proposed for preventing VEGF₁₆₅ binding to neurophilin-1.

First of all, the use of soluble forms of neurophilin-1, capable of binding VEGF₁₆₅ in the extra cellular medium; two soluble forms of neurophilin-1 have been described. These are the forms referred to as S11 and S12, which correspond to splice variants of neurophilin-1 (13). The isoform s₁₂NRP can bind to VEGF₁₆₅. Overexpression of s₁₂NRP in AT2 cells (rat prostate carcinoma) injected into animals decreases tumor development (14).

Moreover, the use of molecules which compete with VEGF₁₆₅ has been proposed, and in particular the use of semaphorin-3A.

Several studies have shown that, in endothelial cells, semaphorin-3A is capable of competing with VEGF₁₆₅ for binding to neurophilin-1. This leads to a decrease in the effect of VEGF₁₆₅ on the proliferation of the cells (15, 16).

It has also recently been described that VEGF₁₆₅ and semaphorin-3A, by competing with one another for binding to neurophilin-1, act on cell migration and retraction (12).

WO 99/29729 describes the use of a neurophilin-1 antagonist, and more particularly of a member of the semaphorin/collapsin family, which has antagonist activity with respect to VEGF₁₆₅, for inhibiting metastases in a patient having malignant tumor cells.

More particularly, WO 99/29729 demonstrates that members of the semaphorin/collapsin family are not only inhibitors of neuronal addressing, but that they are also inhibitors of the mobility of endothelial and tumor cells which express neurophilin-1. The mechanism proposed is that semaphorin-3A competes with VEGF₁₆₅ at the level of neurophilin-1 and thus blocks the angiogenesis-promoting pathway. It is this anti-angiogenic action which is thought to be responsible for the inhibition of tumor development.

Such a method of action of semaphorin-3A will subsequently be referred to as “VEGF pathway”.

In other words, semaphorin-3A had, to date, only been described for its VEGF₁₆₅ antagonist activity. In fact, the growth of a tumor requires the vascularization of said tumor to be sufficient to provide it with the oxygen and the nutrients essential to its development and to its rapid division. Such a vascularization is provided by angiogenesis, which is itself increased by the action of VEGF₁₆₅ bound to neurophilin-1. Blocking this action, by means of a VEGF₁₆₅ antagonist, such as semaphorin-3A, which, by binding to neurophilin-1, prevents VEGF₁₆₅ from doing so, inhibits the angiogenesis and, as a result, decreases the proliferation and migration of the tumor cells.

The present invention therefore relates to a field other than that of the prior art, in the sense that it concerns epithelial cells and not endothelial cells, and that it is based on demonstrating a direct action of semaphorin-3A on the invasiveness of the tumor cells, which action is completely independent of VEGF₁₆₅ and was, until now, unknown.

Thus, the present invention relates to a direct action on the invasion of tumor cells, and not an anti-angiogenic activity. Without wishing to be bound by any theory, the inventors think that semaphorin-3A interacts with neurophilin in a way which is different from VEGF₁₆₅, and causes a different biological response.

More particularly, according to a first aspect, the present invention relates to a method for the in vitro evaluation of the aggressiveness of tumor cells from a tumor sample to be tested, which consists in measuring the level of expression of all or part of the gene encoding semaphorin-3A in normal epithelial cells and in epithelial tumor cells from said sample and in declaring said tumor cells as having a strong invasive power if underexpression of said gene is observed therein.

The term “expression” is intended to mean any mechanism which leads, through transcription of the DNA to various RNAs and translation of the mRNA to proteins, to the decoding of the genetic information contained in the hereditary material. More particularly, the level of expression of a gene can occur at the RNA level or at the peptide/protein level.

An “underexpression” will result in an amount of expression products which is less than that obtained subsequent to normal expression and, conversely, an “overexpression” will result in a greater amount.

The term “all or part” should be understood to mean that the gene may consist either of all the nucleotide sequence encoding semaphorin-3A, or of only part of this sequence, on condition that the sequence conserves the ability to encode said gene (functional homologue). The term “part of the sequence” should be understood to mean a region of said nucleotide sequence lacking one or more terminal nucleotides. It may also be a region of the nucleotide sequence in which one or more nucleotides have been deleted or substituted with other nucleotides.

The present invention therefore envisions quantifying all the products resulting from the expression of the gene encoding semaphorin-3A, for example the RNAs, but also the mRNAs or alternatively the peptides/proteins.

The term “strong invasive power” should be understood to mean a high probability of extracapsular extension and of formation of metastases.

In practice, the method according to the invention comprises the following steps:

i) quantifying, in vitro, the product of expression of all or part of the gene encoding semaphorin-3A within, firstly, said tumor cells and, secondly, said normal cells,

ii) comparing the results obtained in step i), and

iii) declaring the tumor as having strong invasive power if an underexpression by a factor of at least 30, and preferably of at least 10, is observed.

The term “product of expression” should be understood to mean any product, in any form whatsoever, resulting from the mechanism of expression as described above.

The in vitro quantification of the product of expression of all or part of the gene encoding semaphorin-3A can be carried out by any technique known to those skilled in the art, such as, for example, assaying expression of mRNA encoding semaphorin-3A by RT-PCR or else assaying semaphorin-3A using specific antibodies.

Due to the considerable variations between individuals, the reference values are preferably obtained by assaying in cells from normal prostate tissue, derived from the same patient.

Although the present invention applies to all types of cancer, it is directed more particularly toward prostate cells.

According to a second aspect, the present invention also relates to a method for the in vitro evaluation of the effectiveness of an antitumor treatment, which consists in measuring, on a sample of epithelial tumor cells, the level of expression of all or part of the gene encoding semaphorin-3A at predetermined time periods, and in declaring said treatment to be effective if, in the course of the various predetermined time periods, an increase in the expression of all or part of the gene encoding semaphorin-3A is observed.

Thus, it is possible to follow the progression of a given treatment in a patient and, where appropriate, to modify this treatment if the results obtained are not satisfactory.

In a first variant of implementation of the invention, said expression product consists of RNAs and/or cDNAs.

In this case, the present invention also relates to a kit for carrying out the method which is the subject of the invention, which comprises:

a) the primers which hybridize specifically with the RNAs and/or cDNAs derived from all or part of the gene encoding semaphorin-3A, and

b) the buffers and enzymes required for the amplification, labeling and hybridization reactions.

The term “primers” should be understood to mean any DNA oligonucleotide sequence which hybridizes specifically to a complementary single-stranded DNA sequence during the initiation of a replication. The primers used in the present invention are described later.

In practice, the determination of primers is commonly carried out, based on known sequences, using computer programs which are well known to those skilled in the art, such as the Oligo 4 program (National Biosciences, Inc., Plymouth, Minn.).

In a second variant of implementation of the invention, said expression product consists of proteins.

In this case, the present invention also relates to a kit for carrying out the method which is the subject of the invention, which comprises:

a) the antibodies which complex specifically with the proteins derived from all or part of the gene encoding semaphorin-3A,

b) the buffers and enzymes required for the amplification, labeling and hybridization reactions.

According to a third aspect, the present invention envisions the use of semaphorin-3A for the treatment of prostate cancer and, more particularly, a method for inhibiting the invasive power of epithelial tumor cells, which consists in increasing the amount of semaphorin-3A present in, and/or in the vicinity of, said epithelial tumor cells.

As described above, the novelty of such a treatment lies, firstly, in the fact that it targets only the invasiveness of the epithelial tumor cells and, secondly, in the fact that semaphorin-3A acts directly on said epithelial tumor cells, i.e. independently of the “VEGF pathway”.

A first embodiment of the method of treatment consists in increasing the expression of all or part of the endogenous gene encoding semaphorin-3A in epithelial tumor cells and/or in the vicinity of said cells.

The term “endogenous gene” should be understood to mean the gene(s) encoding the semaphorin-3A initially present in each epithelial cell.

A second embodiment of the method of treatment according to the invention consists in introducing, into said epithelial tumor cells and/or in the vicinity of said cells, all or part of a nucleotide sequence encoding semaphorin-3A.

The expression “introducing” should not be taken in the literal sense, but envisions any method which results in the presence of semaphorin-3A in or in the vicinity of epithelial tumor cells. It may equally involve topical introduction or systemic introduction, for example oral introduction.

The term “exogenous gene” should be understood to mean any natural or synthesized gene, or part of a gene, encoding semaphorin-3A, which is not initially present in each epithelial cell.

For example, such an exogenous gene may be in the form of a purified polynucleotide encoding semaphorin-3A, or else in the form of a recombinant vector comprising a polynucleotide encoding semaphorin-3A.

The term “purified polynucleotide” should be understood to mean any polyribonucleotide or poly-deoxyribonucleotide, i.e. any modified or unmodified, double-stranded or single-stranded DNA or RNA which has been isolated or separated from its natural environment, and from which approximately 60%, preferably approximately 75%, and better still approximately 90% of the residues with which it is naturally associated have been removed.

The term “vector” should be understood to mean any viral or nonviral expression system known to those skilled in the art.

The term “recombinant” refers to the results of methods and manipulations in which nucleic acids, or any other biological material, are cleaved, synthesized, combined or otherwise manipulated, in vitro, enzymatically, chemically or biologically in order to obtain the desired products in cells or any other biological system.

A recombinant vector may be a cloning, expression or insertion vector such as a vector of the AdV (adenovirus) type or else the AAV (adeno-associated virus) type.

A third embodiment of the method of treatment according to the invention consists in introducing, into said epithelial tumor cells and/or in the vicinity of said cells, a polypeptide comprising all or part of semaphorin-3A in unmodified form or partially mutated.

A fourth embodiment of the method of treatment according to the invention is based on cell therapy, i.e. the use of any method employing cells in order to treat a tissue or organ failure. More particularly, in the present case, it will involve introducing into or in the vicinity of the organ to be treated cells capable of secreting semaphorin-3A.

The present invention therefore envisions introducing, in the vicinity of epithelial tumor cells, cells capable of secreting semaphorin-3A.

In practice, such cells may consist of cells of the epithelial or fibroblast type capable of secreting semaphorin-3A naturally or after modification of said cells, and may be obtained by any genetic engineering technique known to those skilled in the art, such as the integration of a vector containing a gene encoding semaphorin-3A into the genome of said cells.

A fifth embodiment of the method of treatment according to the invention consists in introducing, into said epithelial tumor cells and/or in the vicinity of said epithelial tumor cells, any substance similar to semaphorin-3A.

The expression “substance similar to semaphorin-3A” should be understood to mean any natural or synthetic substance which has the same properties as semaphorin-3A or, at the very least, its ability to act directly on epithelial tumor cells independently of VEGF₁₆₅. To do this, it must also have the same ability to bind to neurophilin-1.

In a particular embodiment of the present invention, a method for screening substances similar to semaphorin-3A is also envisioned.

To do this, the present invention describes a method for identifying any substance similar to semaphorin-3A, which consists in performing, on epithelial tumor cells, an invasion assay in the absence of VEGF₁₆₅, or in the presence of VEGF₁₆₅ but with the concomitant presence of a substance which blocks the action of said VEGF₁₆₅, and in selecting as analogues the substances which inhibit said invasion.

According to yet another aspect of the present invention, a pharmaceutical composition intended for the treatment of cancers, in particular prostate cancer, and more particularly intended to inhibit the invasiveness of epithelial tumor cells, is envisioned.

More particularly, the present invention envisions a pharmaceutical composition for inhibiting the invasiveness of epithelial tumor cells, which comprises all or part of semaphorin-3A or of the gene encoding said semaphorin-3A.

Another embodiment of the present invention consists of a pharmaceutical composition for inhibiting the invasiveness of epithelial tumor cells, which comprises at least one substance similar to semaphorin-3A.

The invention will be understood more clearly in light of the results from the following experiments, which are divided into two parts, namely a first part affecting the expression of the gene encoding semaphorin-3A and a second part concerning the anti-invasiveness action of semaphorin-3A.

Materials and Methods

I. Measurement of expression of the Gene Encoding Semaphorin-3A

I.1. Patients and Samples

Forty-four primary prostate tumors were analyzed.

Tumor samples were obtained from patients undergoing surgery at the St-Louis Hospital in Paris, La Cavale Blanche Hospital in Brest and the CHU [University Teaching Hospital] in Nancy (France). Thirty-two patients had a clinically localized prostate tumor, seventeen of which were limited to the prostate and fifteen of which had extracapsular extension. Twelve patients had hormone-refractory carcinomas.

Seven normal prostate tissue samples and RNAs derived from a pool of 47 normal human prostate tissues (marketed by Clontech, Palo-Alto, Calif.) were used to assess the basal level of mRNA encoding semaphorin-3A in a normal prostate tissue.

I.2. Selected Tissues

Clinically localized malignant tumor and benign tumor samples were obtained by means of radical prostatectomy, whereas hormone-refractory tumors were obtained by means of transurethral resection.

A part of the selected tissues was immediately placed in liquid nitrogen for nucleic acid extraction, while the adjacent sections were stained with H/E (hematoxylin and eosin) and were examined histologically.

Malignant areas from tumor samples were carefully selected by means of microdissection so as to obtain a homogeneous cell population and thereby avoid any “dilution” of tumor-specific genetic changes with nucleic acids from normal and reactive cells present in the same sample.

For these reasons, a sample is considered to be suitable for molecular studies if the proportion of tumor cells exceeds 90% of epithelial cells. The histological diagnosis, the clinical staging based on the TMM system and the Gleason scores were determined in each case during a work-up after surgery. Twelve were hormone-refractory tumors, whereas, after pathological examination, 17 of the 32 tumors were strictly localized (pT2) and 15 exhibited capsular extension (pT3). The Gleason scores for the carcinomas were 4-6 (7 cases), 7 (24 cases) and 8-10 (13 cases).

Normal prostate tissues were obtained by means of radical prostatectomy, histologically checked and selected with respect to their normality and to the epithelial cell component type.

I.3 Nucleic Acid Extraction

a) RNA Extraction

Total RNA was extracted from tissue samples using acid phenol-guanidium. The quality of the RNA samples was controlled by agarose gel electrophoresis and staining with ethidium bromide. The 18S and 28S RNA bands were visualized under ultraviolet light in order to verify the assessment of the extraction.

b) cDNA Synthesis

RNA was reverse transcribed in a final volume of 20 μl containing 1×RT buffer (500 mM of each dNTP, 3 mM of MgCl₂, 75 mM of KCl, 50 mM of Tris-HCl at pH 8.3), 10 units of Rnasin® ribonuclease inhibitor (Promega, Madison, Wis.), 10 mM of dithiothreitol, 50 units of Superscript II Rnase H⁻ reverse transcriptase (Gibco BRL, Gaithersburgh, Md.), 1.5 mM of random hexamers (Pharmacia, Uppsala, Sweden) and 1 μg of total RNA. The samples were incubated at 20° C. for 10 minutes and at 42° C. for 30 min, and the reverse transcriptase was inactivated by heating at 99° C. for 5 minutes and cooling at 5° C. for 5 minutes.

I.4. Real-Time Quantitative RT-PCR

Quantitative values were obtained from the threshold cycle (Ct) number at which the increase in signal associated with an exponential increase in the PCR product begins to be detected (using PE Biosystems analysis software), according to the manufacturer's manual.

The precise amount of total RNA added to each reaction (based on the optical density) and its quality (i.e. lack of extensive degradation) are both difficult to control. Consequently, transcripts of the RPLPO gene (also known as 36B4) encoding human acidic ribosomal phosphoprotein (PO) were also quantified as endogenous RNA control, and each sample was standardized on the basis of its RPLPO content. A prostate tumor (T30) which contained the smallest accurately quantifiable amount of mRNA encoding semaphorin-3A in the assays was used as calibrator.

The final results, referred to as Nsema3A and expressed as N-fold difference between the relative expression of semaphorin-3A with respect to the RPLPO gene and to the calibrator, were determined as follows:

Nsema3A=2^{(ΔCt(sample)-Δct(calibrator))}

where the ΔCt values for the sample and for the calibrator were determined by subtracting the average Ct value per sample (each sample was tested twice) for the gene encoding semaphorin-3A from the average Ct value for the RPLPO gene. The ratio was standardized such that the mean ratio for the 7 normal prostate samples corresponds to a value of 1.

I.5. Primers and Products for PCR

Primers were chosen with the assistance of the computer programs Oligo 4 (National Biosciences, Plymouth, Minn.) and Primer Express (Perckin-Elmer Applied Biosystems, Foster City, Calif.). BLASTN searches were performed against dbEST and nr (the non-redundant set of GenBank, EMBL and DDBJ sequence databases) to confirm the complete gene specificity of the nucleotide sequences chosen as primers. To avoid amplification of contaminating genomic DNA, a first primer was placed at the junction between two exons and a second was placed in a third exon.

It was checked that, after a PCR reaction, these primers gave a single band on agarose gel; the PCR products were in addition purified and sequenced to confirm the specificity of the primers.

The nucleotide sequences of the primers were as follows:

Semaphorin-3A

| | |
|---|---|
| Upper primer: | 5′-CCTATGAACAATCGCCCAATAGTG-3′ |
| Lower primer: | 5′-CTTTAAGAACGGTCCCAACATCTGT-3′ |

RPLPO

| | |
|---|---|
| Upper primer: | 5′-GGCGACCTGGAAGTCCAACT-3′ |
| Lower primer: | 5′-CCATCAGCACCACAGCCTTC-3′ |

I.6. PCR Amplification

All PCR reactions were performed using an ABI Prism 7000 sequence detection system (Perkin-Elmer Applied Biosystems). The PCR was performed using the SYBR® Green PCR Core reagents kit (Perkin-Elmer Applied Biosystems). The thermal cycling conditions comprised an initial denaturation step at 95° C. for 10 min and 45 cycles comprising 15 seconds at 95° C., followed by 1 minute at 65° C. The experiments were performed in duplicate for each data point.

II. Invasiveness Assays

II.1. Cloning of Semaphorin-3A

The semaphorin-3A cDNA, 2.3 kb in size, was obtained by RT-PCR from the CaHPV10 prostate line. This cDNA was first of all cloned between the EcoRI and XBaI restriction sites of the expression vector pcDNA3.1/Zeo (+) (Invitrogen). It was then modified by PCR in order to delete the translation stop codon, and was finally cloned, in phase with the myc and histidine epitopes, between the EcoRI and XBaI restriction sites of the vector pcDNA4/Myc-His (Invitrogen). The integrity of the cDNA, cloned into the vector pcDNA4/Myc-His, was verified by direct sequencing. Its sequence is identical to that published by Kolodkin et al. (accession no. L26081).

The sequences of the primers used for the amplification are as follows:

5′ primer
5′ CGG AAT TCT GCA GCA TGG GCT GGT TAA CT 3′
3′ primer
5′ GCT CTA GAT CAG ACA CTC CTG GGT GCC CT 3′

The sequences of the primers used to delete the stop codon are as follows:

5′ primer

5′ GGA ACA TGG GTT CAT ACA AAC TCT TCT TA 3′
3′ primer
5′ GC TCT AGA GAC ACT CCT GGG TGC C 3′

II.2. Preparation of Conditioned Medium

Cos-7 cells (ECACC) are seeded in a proportion of 20 000 cells/cm² in RPMI containing 10% FCS, 24 hours before transfection, in a 75 cm³ flask (T75). The cells are transfected either with the vector pcDNA4/Myc-HIS or with this vector containing the semaphorin-3A cDNA. The following mixture is added per T75 flask: 15 μg of DNA in 1.5 ml of serum-free RPMI containing 45 μl of FuGENE 6 (Roche). The cells are incubated for 72 hours at 37° C. in a CO₂ incubator. The supernatant is harvested and then centrifuged twice in order to remove the cell debris.

II.3. Control of the Presence of Semaphorin-3A in the Conditioned Medium by Immunoprecipitation and Western Blotting

1 ml of conditioned medium, harvested 72 hours after cell transfection, is incubated with 2 μg of monoclonal anti-myc antibody (Santa Cruz mouse monoclonal anti-c-Myc sc-40) at 4° C., for 16 hours on a rotary shaker. The complex formed by the antibody is precipitated with 20 μl of sepharaose G beads (Amersham Pharmacia Biotech) diluted in half in PBS. The mixture is incubated for 30 minutes at 4° C. on a rotary shaker. After centrifugation, the pellet containing the Sema3Amyc-antibody-bead complex is taken up in 20 μl of Laemmli Sample Buffer (Biorad) and loaded onto an SDS-7% PAGE gel. In parallel, the Cos-7 cells transfected with empty pcDNA4/Myc-HIS or pcDNA4/Myc-HIS containing semaphorin-3A are lysed in buffer containing 50 mM of Tris, pH 8, 200 mM of NaCl, 1% NP40, 0.5% deoxycholic acid, 0.05% SDS and 2 mM EDTA.

The term “empty” should be understood to mean “comprising no semaphorin-3A”.

Fifty micrograms of proteins from the total extract are loaded onto an SDS-7% PAGE gel. After migration, the proteins are transferred onto nitrocellulose membranes. The membrane is incubated for 1 hour at ambient temperature in PBS containing 5% skimmed milk (Biorad) in order to block the nonspecific sites. The membrane is then incubated for 1 hour at ambient temperature with the anti-myc primary antibody (mouse monoclonal anti c-Myc sc-40, Santa Cruz) diluted to 2 ng/ml in PBS containing 5% skimmed milk. The membrane is rinsed 4 times, each time for 10 minutes, in PSB containing 0.1% tween 20, before being incubated for 1 hour at ambient temperature with the secondary antibody (rabbit anti-mouse HRP, Dako). The rinsing is identical to that described above and the membrane is then incubated for 5 minutes in the ECL Plus reagent (Amersham Pharmacia Biotech). The fluorescence is detected using a Storm detector.

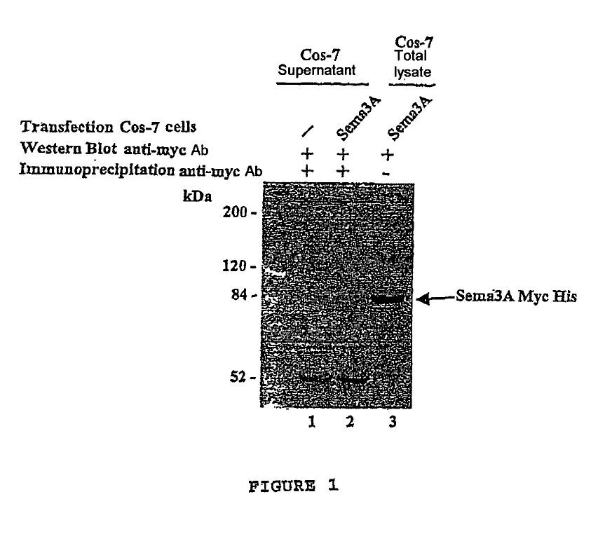

The results are given in FIG. 1, in which:

Column 1: immunoprecipitation and Western blotting with anti-myc antibodies on 1 ml of medium from Cos-7 cells transfected with empty pcDNA4.1,

Column 2: immunoprecipitation and Western blotting with anti-myc antibodies on 1 ml of medium from Cos-7 cells transfected with pcDNA4.1 containing the semaphorin-3A cNDA, and

Column 3: Western blotting performed on 50 μg of total extract of Cos-7 cells.

II.4 Invasion Assays

PC3 cells are placed, in a proportion of 40 000 cells per well, in invasion chambers (BioCoat Matrigel Invasion Chamber, Becton Dickinson, Bedford, Mass.) in conditioned medium (empty pcDNA4/Myc-HIS or pcDNA4/Myc-HIS containing semaphorin-3A). The conditioned medium is also placed in the lower compartment of the chamber.

For the assays carried out in the presence of anti-VEGF₁₆₅ antibody (AF-293-NA, R&D systems), this antibody is added to the conditioned medium at the concentration of 0.5 μg/ml. The cells are incubated for 48 hours at 37° C. in a CO₂ incubator and the noninvasive cells are removed from the upper compartment of the invasion chamber. The invasive cells have digested the matrigel and passed through the 8 μm pores of the membrane and adhere to the lower surface of the membrane. In order to quantify the invasive cells, they are stained with a solution containing 0.5% crystal violet in 25% methanol. The membrane is then cut up and mounted between a slide and cover slip. The cells are then quantified by observation under a microscope.

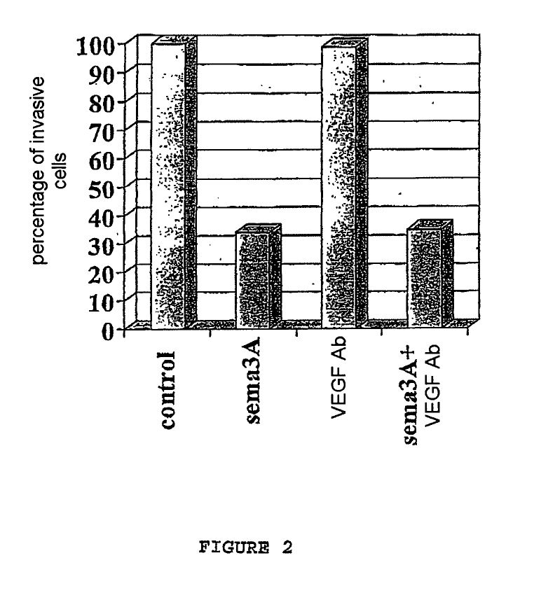

The results are given in FIG. 2, in which:

Column 1: Conditioned medium originating from Cos-7 cells transfected with empty pcDNA4.1 in the absence of VEGF₁₆₅,

Column 2: Conditioned medium originating from Cos-7 cells transfected with pcDNA4 containing the semaphorin-3A cDNA and in the absence of VEGF₁₆₅,

Column 3: Conditioned medium originating from Cos-7 cells transfected with empty pcDNA4.1 and in the presence of the anti-VEGF₁₆₅ antibody,

Column 4: Conditioned medium originating from Cos-7 cells transfected with pcDNA4 containing the semaphorin-3A cDNA in the presence of the anti-VEGF₁₆₅ antibody.

Results

I. Measurement of Expression of the Gene Encoding Semaphorin-3A

The relative expression levels of semaphorin-3A were quantified in 44 malignant tumors, 7 matched with normal prostate tissues, and the RNAs from a pool of 47 normal human prostate tissues (Clontech). The expression levels were determined in the form of ratios between semaphorin-3A and the reference RPLPO gene in order to correct variations in the amount of RNA.

For an individual sample, an underexpression is considered to be significant if the expression level is less than the average value of the semaphorin-3A expression in the normal samples minus 3 times the standard deviation.

Twenty-four of the 32 clinically localized tumor samples and 6 of the 12 hormone-refractory tumor samples show underexpression of semaphorin-3A.

Overall, the results show a significant decrease in semaphorin-3A expression in the prostate tumor samples compared to the normal prostate samples (p<0.001).

Semaphorin-3A thus appears to be underexpressed by a factor of approximately 10 in the tumor samples compared to the normal tissue.

II. Invasiveness Assays

The semaphorin-3A cDNA was cloned in phase with the myc and histidine epitopes of the vector pcDNA4/Myc-HIS. The protein produced therefore contains the myc and his epitopes. A cell culture medium containing the soluble form of semaphorin-3A was prepared by transfecting Cos-7 cells. Seventy-two hours after transfection, the medium is harvested and used for the invasion experiments. The presence of semaphorin-3A was detected, by immunoprecipitation and Western blotting, in the supernatant and in the cell extracts from the transfected Cos-7 cells (FIG. 1, columns 2 and 3). As a control, it was shown that the supernatant from Cos-7 cells transfected with the vector pcDNA4/Myc-HIS contains no semaphorin-3A (FIG. 1, column 1).

The invasion experiments were carried out with PC3 prostate tumor cells and showed that these cells overexpress neurophilin and express very small amounts of semaphorin-3A, which is one of its ligands. An attempt was made to determine whether the restoration of semaphorin-3A expression could play a role in inhibiting the tumoral properties of the PC3 cells. For this purpose, the invasion experiments were carried out in chambers containing matrigel deposited onto a membrane having 8 μm micropores. The cells were placed in the upper compartment of the chamber in culture medium, the lower compartment also containing medium. Invasive cells digest the matrigel and migrate through the pores into the lower compartment. The experiments were carried out in the presence of conditioned medium prepared, firstly, from supernatant from Cos-7 cells transfected with the vector pcDNA4/Myc-HIS (control medium) and, secondly, from supernatant from Cos-7 cells transfected with the vector pcDNA4/Myc-HIS containing the semaphorin-3A cDNA. Two independent experiments were carried out in triple for each condition. The invasive cells under the membrane were quantified by microscopy and, for each invasion chamber, two different fields were quantified. The results are given in the form of percentages relative to the number of invasive cells in the empty or control medium: it is noted that the presence of semaphorin-3A in the medium inhibits the invasive capacity of the PC3 cells by 60 to 70% (FIG. 2, columns 1 and 2). The inventors showed that the PC3 cells secrete small amounts of VEGF₁₆₅. In order to confirm the role of semaphorin-3A independently of the VEGF₁₆₅ pathway, invasion experiments in the presence of an antibody which blocks the action of the VEGF₁₆₅ secreted by the PC3 cells were carried out. The antibody, added to the conditioned medium at the concentration of 0.5 μg/ml, does not modify the invasion of the PC3 cells, whether in the absence (FIG. 2, column 3) or in the presence of semaphorin-3A in the conditioned medium (FIG. 2, column 4). The inhibitory role of semaphorin-3A on the invasive capacity of the PC3 cells is therefore shown in this way. It was also demonstrated that the VEGF₁₆₅ secreted by the PC3 cells has no effect on the invasiveness of the cells, since blocking the VEGF₁₆₅ with a specific antibody does not modify the invasiveness of the cells. Consequently, semaphorin-3A indeed has a direct and agonistic role which strongly inhibits the invasive power of epithelial tumor cells.

### REFERENCES

1. Cuzin B. & Thoral J E., “Opportunité d'un dépistage systématique du cancer de la prostate par le dosage de l'antigène spécifique de la prostate” [Opportunity for systematic screening of prostate cancer by assaying prostate-specific antigen], Document of the Agence Nationale d'Accréditation et d'Evaluation en Santé (ANAES) [National Agency for Habilitation and Evaluation in Public Heath], 121 pages (1998)

2. Grosclaude P., Menegoz F., Schaffer P., Macé Lesec'h J., Avreux P., Lemab G. & al., “Dépistage du cancer de la prostate (II). Le cancer de la prostate est-il un problème de santé publique? Actualisation des chiffres d'incidence et de mortalité en France de 1982 à 1990” [Screening prostate cancer (II). Is prostate cancer a public health problem? Updating of figures for incidences and mortality in France from 1982 to 1990], Progrès Urol., 7/647-54 (1997)

3. World Health Organization, International Agency for Research on Cancer & International Association of Cancer Registries, “Age-standardized and cumulative incidence: rates and standard errors. Prostate (ICD-9 185)”, IARC, pp. 970-1 (1992)

4. Neufeld G, Cohen T, Gengrinovitch S, Poltorak Z., “Vascular endothelial growth factor (VEGF) and its receptors”, FASEB J. 1999 January; 13 (1): 9-22.

5. Takahashi T, Fournier A, Nakamura F, Wang L H, Murakami Y, Kalb R G, Fujisawa H, Strittmatter S M., “Plexin-neuropilin-1 complexes form functional semaphorin-3A receptors”, Cell. 1999 Oct. 1;99(1): 59-69.

6. Tamagnone L, Artigiani S, Chen H, He Z, Ming G I, Song H, Chedotal A, Winberg M L, Goodman C S, Poo M, Tessier-Lavigne M, Comoglio P M., “Plexins are a large family of receptors for transmembrane, secreted, and GPI-anchored semaphorins in vertebrates”, Cell. 1999 October 1;99(1): 71-80.

7. Liu B P, Strittmatter S M., “Semaphorin-mediated axonal guidance via Rho-related G proteins”, Curr Opin Cell Biol. 2001 October; 13(5): 619-26.

8. Pasterkamp R J, Verhaagen J., “Emerging roles for semaphorins in neural regeneration”, Brain Res Brain Res Rev. 2001 March; 35(1): 36-54.

9. Soker S, Takashima S, Miao H Q, Neufeld G, Klagsbrun M., “Neuropilin-1 is expressed by endothelial and tumor cells as an isoform-specific receptor for vascular endothelial growth factor”, Cell. 1998 Mar. 20;92(6): 735-45.

10. Fuh G, Garcia K C de Vos A M., “The interaction of neuropilin-1 with vascular endothelial growth factor and its receptor flt-1”, J Biol. Chem. 2000 Sep. 1;275(35): 26690-5.

11. Ortega N, Hutchings H, Plouet J., “Signal relays in the VEGF system”, Front Biosci. 1999 Feb. 1;4: D141-52.

12. Soker S., “Neuropilin in the midst of cell migration and retraction”, Int J Biochem Cell Biol. 2001 April; 33(4): 433-7.

13. Rossignol M, Gagnon M L, Klagsbrun M., “Genomic organization of human neuropilin-1 and neuropilin-2 genes: identification and distribution of splice variants and soluble isoforms”, Genomics. 2000 Dec. 1;70(2): 211-22.

14. Gagnon M L, Bielenberg D R, Gechtman Z, Miao H Q, Takashima S, Soker S, Klagsbrun M., “Identification of a natural soluble neuropilin-1 that binds vascular endothelial growth factor: In vivo expression and antitumor activity.”, Proc Natl Acad Sci USA. 2000 Mar. 14;97(6): 2573-8.

15. Miao H Q, Soker S, Feiner L, Alonso J L, Raper J A, Klagsbrun M., “Neuropilin-1 mediates collaspin-1/semaphorin III inhibition of endothelial cell motility: functional competition of collapsin-1 and vascular endothelial growth factor-165”, J. Cell Biol. 1999 Jul. 12;146(1): 233-42.

16. Shima D T, Mailhos C., “Vascular developmental biology: getting nervous.”, Curr Opin Genet Dev. 2000 October; 10(5): 536-42.

1. A method for the in vitro identification of tumor cells in a tumor sample to be tested, characterized in that it consists in measuring the level of expression of all or part of the gene encoding semaphorin-3A in normal epithelial cells from said sample and in epithelial cells, from this same sample, suspected of being tumor cells, and in declaring these suspected cells to be effectively tumor cells if an underexpression of said gene is observed therein compared to the expression observed in the normal cells.

2. The method as claimed in clam 1 applied to the evaluation of the aggressiveness of tumor cells, characterized in that it comprises the following steps:
i) quantifying, in vitro, the product of expression of all or part of the gene encoding semaphorin-3A within, firstly, said tumor cells and, secondly, said normal cells,
ii) comparing the results obtained in step i), and
iii) declaring the tumor as having strong invasive power if an underexpression by a factor of at least 3 is observed.

3. The method as claimed in claim 2, characterized in that said tumor is declared as having strong invasive power if an underexpression by a factor of at least 10 is observed.

4. The method as claimed in one of claims 1 to 3, characterized in that said tumor cells consist of prostate cells.

5. A method for the in vitro evaluation of the effectiveness of an antitumor treatment, characterized in that it consists in measuring, on a sample of epithelial tumor cells, the level of expression of all or part of the gene encoding semaphorin-3A at predetermined time periods, and in declaring said treatment to be effective if, in the course of the various predetermined time periods, an increase in the expression of all or part of the gene encoding semaphorin-3A is observed.

6. The method as claimed in any one of claims 2 to 5, characterized in that said expression product consists of RNAs and/or cDNAs.

7. A kit for carrying out the method as claimed in claim 6, characterized in that it comprises:
a) the primers which hybridize specifically with the RNAs and/or cDNAs derived from all or part of the gene encoding semaphorin-3A, and
b) the buffers and enzymes required for the amplification, labeling and hybridization reactions.

8. The method as claimed in any one of claims 2 to 5, characterized in that said expression product consists of proteins.

9. A kit for carrying out the method as claimed in claim 8, characterized in that it comprises:
a) the antibodies which complex specifically with the proteins derived from all or part of the gene encoding semaphorin-3A,
b) the buffers required for the reactions for revealing the formation of the complexes and/or for quantifying these complexes.

10. The use of a polypeptide comprising all or part of semaphorin-3A, for producing a pharmaceutical composition intended to inhibit the invasive power of epithelial tumor cells.

11. The use of a nucleic acid comprising all or part of the gene encoding semaphorin-3A, for producing a pharmaceutical composition intended to inhibit the invasive power of epithelial tumor cells.

12. A method for identifying any substance similar to semaphorin-3A, characterized in that it consists in performing, on epithelial tumor cells, an invasion assay in the absence of VEGF₁₆₅, or in the presence of VEGF₁₆₅ but with the concomitant presence of a substance which blocks the action of said VEGF₁₆₅, and in selecting as analogues the substances which inhibit said invasion.

13. The use of any substance similar to semaphorin-3A, for producing a pharmaceutical composition intended to inhibit the invasive power of epithelial tumor cells.

14. A pharmaceutical composition for inhibiting the invasiveness of epithelial tumor cells, characterized in that it comprises all or part of semaphorin-3A or of the gene encoding said semaphorin-3A.

15. A pharmaceutical composition for inhibiting the invasiveness of epithelial tumor cells, characterized in that it comprises at least one substance similar to semaphorin-3A.


SEQUENCE LISTING (8 sequences)

SEQ ID NO: 1 - DNA, 24 bp, Unknown
  Upper primer of semaphorine-3A chosen with the assistance of programs Oligo4 (National Biosciences, Plymouth, MN) et Primer express (Perkin - Elmer Applied Biosystems, Foster city, CA)
cctatgaacaatcgcccaatagtg

SEQ ID NO: 2 - DNA, 25 bp, Unknown
  Lower primer of semaphorin-3A chosen with the assistance of programs Oligo4 (National Biosciences, Plymouth, MN) and Primer express (Perkin-Elmer Applied Biosystems, Foster city, CA)
ctttaagaacggtcccaacatctgt

SEQ ID NO: 3 - DNA, 20 bp, Unknown
  Upper primer of RPLPO chosen with the assista nce of programs Oligo4 (National    Biosciences, Plymouth, MN) and Primer express (Perkin- Elmer Applied Biosystems, Foster city, CA)
ggcgacctggaagtccaact

SEQ ID NO: 4 - DNA, 20 bp, Unknown
  Lower primer of RPLPO chosen with the assista nce of programs Oligo4 (National    Biosciences, Plymouth, MN) and Primer express(Perkin- Elmer Applied Biosystems, Foster city, CA)
ccatcagcaccacagccttc

SEQ ID NO: 5 - DNA, 29 bp, Unknown
  5′ primer of semaphorin-3A
cggaattctgcagcatgggctggttaact

SEQ ID NO: 6 - DNA, 29 bp, Unknown
  3′ primer of semaphorin-3A
gctctagatcagacactcctgggtgccct

SEQ ID NO: 7 - DNA, 29 bp, Unknown
  5′ primer used to delete the stop codon of translation of ADNc   of semaphorin-3A
ggaacatgggttcatacaaactcttctta

SEQ ID NO: 8 - DNA, 24 bp, Unknown
  3′ primer used to delete the stop codon of translation of ADNc   of semaphorin-3A
gctctagagacactcctgggtgcc